(19) United States
(12) Patent Application Publication
(10) Pub. No.: US 2026/0102281 A1
(43) Pub. Date: Apr. 16, 2026

(54) NEEDLE INJECTION SYSTEM WITH BACKPRESSURE SENSING

(71) Applicant: West Pharmaceutical Services, Inc., Exton, PA (US)

(72) Inventors: Bingxi Yan, Downingtown, PA (US); Anu Prabhathachandran, Phoenixville, PA (US); Menachem Peretz, Ra'anana (IL); Michael Woodburn, Ardmore, PA (US)

(21) Appl. No.: 19/352,858

(22) Filed: Oct. 8, 2025

Related U.S. Application Data
(60) Provisional application No. 63/705,934, filed on Oct. 10, 2024.

(51) Int. Cl. A61F 9/00; A61M 5/48

(52) U.S. Cl. CPC A61F 9/0017; A61M 5/486; A61M 2205/3344; A61M 2210/0612

(57) ABSTRACT

A system for injecting medicament into a target region includes a syringe with a barrel to contain the medicament, a movable plunger within the barrel, and a needle in fluid communication with the barrel that has a tip. The system includes a sensor configured to sense backpressure exerted on the plunger. A controller is then used to determine satisfaction of a needle advancement criteria based on the sensed backpressure. The needle advancement criteria being associated with the tip of the needle extending through target tissue into the target region.

### CROSS REFERENCE TO RELATED APPLICATIONS

This application claims the benefit of U.S. Provisional Application No. 63/705,934, filed Oct. 10, 2024, the entirety of which is incorporated herein for any and all purposes.

### BACKGROUND

Intraocular drug injection is a medical procedure used to deliver medicament (e.g., therapeutic agents) directly into the eye. Intraocular drug injection can target, for example, the vitreous humor or other intraocular structures. Intraocular drug injection can be effective for treating various ocular conditions where localized drug delivery is crucial for efficacy, such as retinal vein occlusion, diabetic retinopathy, age-related macular degeneration, among others. Intraocular drug injection can involve the use of a fine needle to inject medication through the sclera, ensuring precise delivery to the affected area while minimizing systemic exposure and potential side effects.

### BRIEF SUMMARY

Aspects of this disclosure are directed to systems and processes for injecting medicament into a target region, such as for example the suprachoroidal space between the sclera and the choroid. Though the systems and processes are suitable for injecting medicament into the suprachoroidal space, the systems and processes are not limited to ocular injections and can be used to inject medicament into other target regions including, for example, the epidural space surrounding spinal nerves, among other possibilities. The systems can include a syringe with a barrel containing the medicament, a plunger movable within the barrel, and a needle in fluid communication with the barrel. The systems can also include a sensor configured to sense a backpressure exerted on the plunger. The system can also include a controller configured, when the tip of the needle extends through a target tissue and into the target region, to determine satisfaction of a needle advancement criteria based on the sensed backpressure. By determining satisfaction of the needle advancement criteria in this manner, the systems and processes can more accurately sense when the needle has reached the target region. Moreover, because the sensed backpressure exerted on the plunger can be used to determine satisfaction of the needle advancement criteria, the incidence of dimpling (and patient discomfort associated therewith) can be reduced or eliminated since the systems and methods of this disclosure can administer the medicament to the target region without applying pressure to the conjunctive using the hub of the needle.

One general aspect of this disclosure includes a system for injecting medicament into a target region. The system includes a syringe. The syringe can include a barrel configured to contain the medicament, a plunger movable within the barrel, and a needle in fluid communication with the barrel. The needle may include a tip. The system also includes a sensor configured to sense a backpressure exerted on the plunger. The system also includes a controller configured, when the tip of the needle extends through a target tissue and into the target region, to determine satisfaction of a needle advancement criteria based on the sensed backpressure.

Implementations may include one or more of the following features. The controller is configured to determine satisfaction of the needle advancement criteria based on the sensed backpressure changing from within a range of threshold backpressures to outside the range of threshold backpressures. The controller is configured to determine satisfaction of the needle advancement criteria based on the sensed backpressure falling from within the range of the range of threshold backpressures to below the range of threshold backpressures. The system further may include a needle actuator configured to drive the needle distally relative to the barrel, and the controller is configured to control the needle actuator based on satisfaction of the needle advancement criteria. The controller is configured, when the tip of the needle is in contact with the target tissue and the sensed backpressure is within the range of threshold backpressures, to determine that the needle advancement criteria is not satisfied, and the controller is configured, in response to the determination that the needle advancement criteria is not satisfied, to control the needle actuator to drive the needle distally relative to the barrel until the needle advancement criteria is satisfied. The system further may include a human machine interface, and the controller is configured, in response to a determination of satisfaction of the needle advancement criteria, to control the human machine interface to indicate that the needle advancement criteria is satisfied. The controller is configured, when the tip of the needle is in contact with the target tissue and the sensed backpressure is within a range of threshold backpressures, to determine satisfaction of a plunger advancement criteria. The system further may include a plunger actuator configured to drive the plunger to cause the plunger to move distally within the barrel, and the controller is configured to control the plunger actuator based on satisfaction of the plunger advancement criteria. The controller is configured, when the tip of the needle is in contact with the target tissue and the sensed backpressure is outside the range of threshold backpressures, to determine that the plunger advancement criteria is not satisfied, and the controller is configured, in response to the determination that the plunger advancement criteria is not satisfied, to control the plunger actuator to drive the plunger distally until the plunger advancement criteria is satisfied. The system further may include a needle actuator configured to drive the needle distally relative to the barrel, and the controller is configured to control the needle actuator based on satisfaction of the needle advancement criteria. The controller is configured to determine satisfaction of the needle advancement criteria based the sensed backpressure changing from being within the range of threshold backpressures to outside the range of threshold backpressures, the controller is configured, when the tip of the needle is in contact with the target tissue and the sensed backpressure is within the range of threshold backpressures, to determine that the needle advancement criteria is not satisfied, and the controller is configured, in response to the determination that the needle advancement criteria is not satisfied, to control the needle actuator to drive the needle distally relative to the barrel until the needle advancement criteria is satisfied. The plunger advancement criteria is a first plunger advancement criteria, and the controller is configured to determine, when the tip of the needle is within the target region and the plunger contacts a distal end of the barrel, satisfaction of a second plunger advancement criteria in response to a rise in the sensed backpressure. The system further may include a human machine interface, and the controller is configured, in response to a determination of satisfaction of the at least one of the needle advancement criteria, the first plunger advancement criteria, or the second plunger advancement criteria, to control the human machine interface to indicate that the at least one of the needle advancement criteria, the first plunger advancement criteria, or the second plunger advancement criteria is satisfied. The range of threshold backpressures is between 5 kPa and 28 kPa, inclusive. The target tissue is sclera tissue and the target region is a suprachoroidal space.

Another general aspect of this disclosure includes a method of injecting medicament into a target region. The method includes determining, based on sensed backpressure associated with a back pressure exerted on a plunger of a syringe, that a needle advancement criteria is not satisfied when a tip of a needle of the syringe extends into a target tissue. The method also includes driving, in response to the determination that the needle advancement criteria is not satisfied, the needle into the target tissue until the tip of the needle reaches a target region. The method also includes determining, based on the sensed backpressure exerted on the plunger of the syringe, that the needle advancement criteria is satisfied after the tip of the needle is in the target region. The method also includes stopping, in response to the determination that the needle advancement criteria is satisfied, the driving of the needle. The method also includes injecting the medicament within the target region.

Implementations may include one or more of the following features. Determining that the needle advancement criteria is not satisfied is based on the sensed backpressure being within a range of threshold backpressures, and determining that the needle advancement criteria is satisfied is based on the sensed backpressure changing from within the range of threshold backpressures to outside of the range of threshold backpressures. The method may include indicating that the needle advancement criteria is satisfied in response to determining that the needle advancement criteria is satisfied. The method may include: determining, based on the sensed backpressure exerted on the plunger of the syringe, before determining that the needle advancement criteria is not satisfied, that a plunger advancement criteria is not satisfied; driving, in response to determining that the plunger advancement criteria is not satisfied, the plunger distally with the tip of the needle in contact with the target tissue; and determining, based on the sensed backpressure exerted on the plunger of the syringe, after driving the plunger distally, that the plunger advancement criteria is satisfied; and stopping, in response to determining that the plunger advancement criteria is satisfied, the driving of the plunger. Determining that the plunger advancement criteria is not satisfied is based on the sensed backpressure exerted on the plunger of the syringe being outside a range of threshold backpressures, and determining that the plunger advancement criteria is satisfied is based on the sensed backpressure exerted on the plunger of the syringe being within the range of threshold backpressures. Driving of the plunger may include controlling a plunger actuator to drive the plunger distally, and stopping the driving of the plunger may include controlling the plunger actuator to stop the drive of the plunger. The method may include indicating that the plunger advancement criteria is satisfied in response to determining that the plunger advancement criteria is satisfied.

Various additional features and advantages of this invention will become apparent to those of ordinary skill in the art upon review of the following detailed description of the illustrative embodiments taken in conjunction with the accompanying drawings.

### BRIEF DESCRIPTION OF THE SEVERAL VIEWS OF THE DRAWINGS

The following detailed description is better understood when read in conjunction with the appended drawings. For the purposes of illustration, examples are shown in the drawings; however, the subject matter is not limited to the specific elements and instrumentalities disclosed. In the drawings:

FIG. 1A illustrates a schematic view of an intravitreal medicament administration.

FIG. 1B illustrates a schematic view of a subretinal medicament administration.

FIG. 1C illustrates a schematic view of a suprachoroidal medicament administration.

FIG. 2 schematic view of a system for injecting medicament into a target region in accordance with one embodiment.

FIG. 3 illustrates a first embodiment of a process of injecting medicament into a target region.

FIG. 4 illustrates a second embodiment of a process of injecting medicament into a target region.

FIG. 5 illustrates a third embodiment of a process of injecting medicament into a target region.

FIG. 6 illustrates a fourth embodiment of a process of injecting medicament into a target region.

FIG. 7 illustrates a computing system in accordance with aspects of this disclosure.

### DETAILED DESCRIPTION

Retinal diseases are a leading cause of preventable blindness in the world. By 2030, it is estimated that more than 15 million patients in the United States will be living with retinal diseases. Current treatment options for retinal diseases include intravitreal medicament administration, subretinal medicament administration, and suprachoroidal medicament administration.

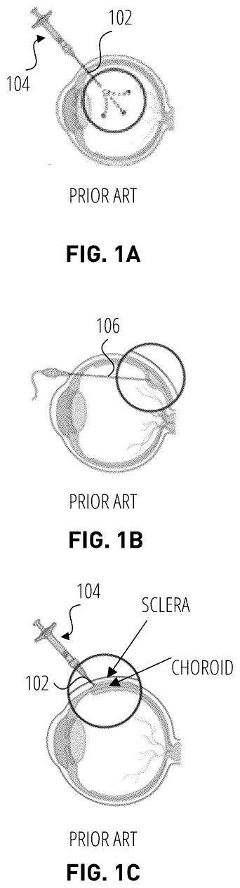

FIG. 1A shows a schematic view of an intravitreal medicament administration. Intravitreal medicament administration can involve injecting medicament directly into the gel-like substance within the vitreous cavity of the eye between the lens and the retina. Intravitreal medicament administration can be performed once every 28-35 days through the lifetime of the patient. Intravitreal medicament administration can be performed by a clinician in an office setting. Intravitreal medicament administration can involve a preparation step, in which the eye and eyelids can be anesthetized using drops or gel to minimize discomfort. The area can then be cleaned with an antiseptic solution. Intravitreal medicament administration can next involve an injection step, in which a needle 102 of a syringe 104 extends through the eye and into the vitreous cavity to inject the medicament. After the injection, the eye can be cleaned again, and the patient can be monitored for complications. Intravitreal medicament administration can used to treat various retinal conditions such as age-related macular degeneration, diabetic retinopathy, and retinal vein occlusion. Drawbacks of intravitreal medicament administration include that intravitreal medicament administration can be non-targeted and can be less advantageous for cell or gene therapy administration than other procedures for administering medicament to a retina.

One goal of retinal medicament administration is to improve the durability of the therapies to reduce the injection frequency for patients. Certain medicaments can provide the option of treat and extend (T&E) regimens allowing the interval between injections to be as far as 16 weeks. T&E regimens can offer a proactive, personalized treatment approach and are associated with a reduced burden for patients and healthcare systems when compared with conventional monthly injection regimens while also optimizing visual outcomes. Furthermore, the predictability of T&E regimens can help to manage clinic flows, which can aid capacity planning. T&E regimen can involve more targeted medicament delivery to the retina, such as via subretinal medicament administration or suprachoroidal medicament administration.

FIG. 1B shows a schematic view of a subretinal medicament administration. Subretinal medicament administration can involve injecting the medicament directly into the subretinal space between the neurosensory retina and the retinal pigment epithelium. In this manner, subretinal medicament administration can be more targeted than intravitreal medicament administration. Subretinal medicament administration can be performed by a clinician in a controlled surgical setting. A typical subretinal medicament administration can involve a preparation step, in which the patient is anesthetized and the eye is cleaned with an antiseptic solution. The typical subretinal medicament administration can next involve an injection step, in which the medicament is injected into the subretinal space via a cannula 106. After the injection, the eye can be monitored for complications and the patient can be prescribed various medications to manage inflammation and/or prevent infection. Subretinal medicament administration can be used to treat various retinal conditions such as, for example, inherited retinal diseases and certain types of macular degeneration. Subretinal medicament administration can be useful for gene therapy treatments. One drawback of subretinal medicament administration is that it is a complex procedure that must be administered by a highly trained professional in a controlled setting.

FIG. 1C shows a schematic view of a suprachoroidal medicament administration using a known syringe 104. Suprachoroidal medicament administration can involve injecting medicament into the suprachoroidal space between the sclera and the choroid. Suprachoroidal medicament administration can provide more targeted delivery of the medicament to the retina, retinal pigment epithelium, and choroid, providing higher bioavailability to these tissues as compared to intravitreal medicament administration. This is because the suprachoroidal space can expand to accommodate the medicament and can provide a more direct path to these tissues than the vitreous cavity, for example. A typical suprachoroidal medicament administration can be performed in an office setting and can involve a preparation step, in which the patient is anesthetized and the eye is cleaned with an antiseptic solution. The typical suprachoroidal medicament administration can next involve an injection step, in which a needle 102 of syringe 104 is inserted into the suprachoroidal space without damaging the retina or choroid. The needle 102 used in typical suprachoroidal medicament administration can a microneedle, which can be less painful for a patient than larger needles. Medicament from within the syringe 104 can be injected via the needle 102 into the suprachoroidal space. Post-injection, the eye can be monitored for complications. Subretinal medicament administration can be used to treat various retinal conditions such as, for example, inherited retinal diseases and certain types of macular degeneration. Subretinal medicament administration can also be useful for gene therapy treatments.

One drawback of current suprachoroidal medicament administration is that it can be difficult to access the suprachoroidal space between the sclera and the choroid with current syringes 104. The sclera is denser than the choroid and therefore the force required to cause the needle 102 to penetrate the sclera is greater than the force required to penetrate the choroid. If too much force is applied, the needle 102 can penetrate through the suprachoroidal space and into the choroid, which can compromise the efficacy of the medicament delivery since the some or all of the medicament intended for the suprachoroidal space would instead be injected into the choroid. In traditional suprachoroidal medicament administration, the clinician gauges that the needle has reached the suprachoroidal space based on a feeling of loss of resistance. This can be subjective and can require significant training to reliably reach the suprachoroidal space.

One way to address over penetration of the needle 102 is to fix the length of the needle 102 to limit the depth that the needle 102 can penetrate into the eye. However, the thickness of the sclera can vary from patient to patient and therefore fixing the length of the needle 102 can cause under penetration issues for some patients with thicker sclera. Under penetration can also compromise the efficacy of the medicament delivery.

Additionally, current suprachoroidal medicament administration can involve applying external pressure onto the conjunctiva with the hub of the needle or with the user's palm or fingers around the penetration site (referred throughout this application as “dimpling”) and starting injection of the medication when the surgeon feels a loss of resistance within the dimpled area, which is indicative of the needle having crossed the denser sclera and entering the relatively less dense suprachoroidal space. Dimpling can cause significant patient discomfort. These technical challenges pose significant pain points in the clinic for administering suprachoroidal injections and compromise the scalability of such procedures.

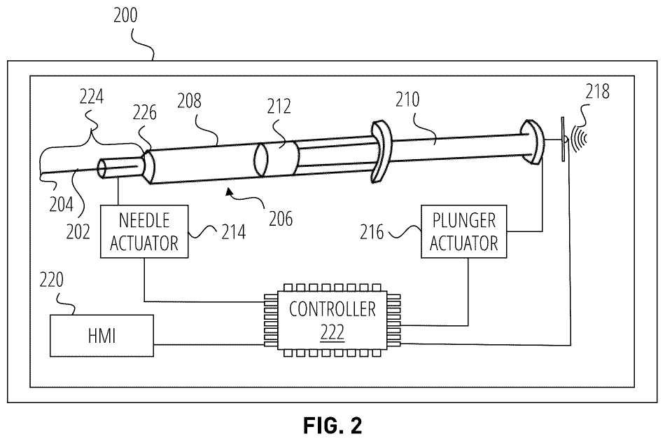

Aspects of this disclosure are directed to systems and processes for injecting medicament into a target region, such as, for example, the suprachoroidal space, which can overcome the technical challenges posed by current suprachoroidal medicament administration. Though the systems and processes are suitable for injecting medicament into the suprachoroidal space, the systems and processes are not limited to ocular injections and can be used to inject medicament into other target regions including for example the epidural space surrounding spinal nerves, among other possibilities. The systems can include a syringe with a barrel containing the medicament, a plunger movable within the barrel, and a needle in fluid communication with the barrel. The systems can also include a sensor configured to sense a backpressure exerted on the plunger. The system can also include a controller configured, when the tip of the needle extends through a target tissue and into the target region, to determine satisfaction of a needle advancement criteria based on the sensed backpressure. By determining satisfaction of the needle advancement criteria in this manner, the systems and processes can more accurately sense when the needle has reached the target region. Moreover, because the sensed backpressure exerted on the plunger can be used to determine satisfaction of the needle advancement criteria, the incidence of dimpling can be reduced or eliminated since the systems and methods of this disclosure can administer the medicament to the target region without applying pressure to the conjunctiva using the hub of the needle. These and other aspects of this disclosure are depicted in FIG. 2-7 and described as follows. Using this system can also eliminate the need for the user to physically apply dimpling pressure to the eye with hands or fingers, which also reduces the discomforts associated with dimpling and reduces chances of introducing foreign objects into the eye afflicting damage to the eye.

FIG. 2 shows a schematic view of a system 200 for injecting medicament into a target region of a patient. The system 200 can include a syringe 206. The syringe 206 can include a needle 202 and a barrel 208 that can contain medicament therein. The needle 202 can be a hollow and can be in fluid communication with the barrel 208. In embodiments, the needle 202 can be a microneedle. A microneedle can be a micron-scale needle with one or more dimensions (e.g., diameter, width, length, etc.) of less than about 1 mm (1000 μm). Though the needle 202 of this disclosure can be a micron-scale needle, the needle 202 can additionally or alternatively include one or more dimensions (e.g., diameter, width, length, etc.) greater than 1000 μm such as dimensions between 1000 μm and 1500 μm, inclusive (i.e., including both endpoints of the range), greater than 1500 μm, among other possibilities.

The syringe 206 can include a plunger 210 that can move proximally towards or distally away from the needle 202. The syringe 206 can further include a piston 212 directly or indirectly connected to the plunger 210. In embodiments, the piston 212 can be integral with the plunger 210. Movement of the plunger 210 distally towards the needle 202 can cause the piston 212 to move distally towards the needle 202. The piston 212 can form a fluid-tight seal with an inner wall of the barrel 208. Distal movement of the piston 212 towards the needle 202 can therefore cause the piston 212 to push the medicament towards the needle 202 and eject the medicament from the syringe 206.

In embodiments, the needle 202 can be moveably connected to barrel 208 such that a distance 224 between a tip 204 of the needle 202 and a distal end 226 of the barrel 208 can be varied. In embodiments, the system 200 can include a needle actuator 214 connected to the needle 202 that can extend or retract the needle 202 relative to the barrel 208 to vary the distance 224. The needle actuator 214 can be any actuator capable of extending or retracting the needle 202 such as, for example, a micro linear actuator, a voice coil actuator, an electrical linear actuator, another type of linear actuator, a manual actuator (e.g., a ratchet), among other possibilities. In some embodiments, the energy that drives the needle actuator 214 can be drawn from the system 200, such as from a power source (e.g., a power supply 714 described later) of the system 200. Additionally, or alternatively, in embodiments a user can manually supply the power to drive the needle actuator 214. For example, the needle actuator 214 can include a ratchet that can be manually operated to extend or retract the needle 202, e.g., in set increments. In alternative embodiments, the needle 202 can be fixedly connected to the barrel 208 such that the distance 224 between the tip 204 and the distal end 226 of the barrel 208 is fixed. In such embodiments, the system 200 can be provided without the needle actuator 214.

In embodiments, the system 200 can include a plunger actuator 216 connected to the plunger 210 that can move the plunger 210 distally and, in embodiments, proximally. The plunger actuator 216 can be any actuator capable of moving the plunger 210 distally and proximally such as, for example, a micro linear actuator, a voice coil actuator, an electrical linear actuator, another type of linear actuator, a manual actuator (e.g., a ratchet), among other possibilities. In some embodiments, the energy that drives the plunger actuator 216 can be drawn from the system 200, such as from the power source (e.g., the power supply 714 described later) of the system 200. Additionally, or alternatively, in embodiments a user can manually supply the power to drive the plunger actuator 216. For example, the plunger actuator 216 can include a ratchet that can be manually operated to move the plunger 210 distally and, in embodiments, proximally, e.g., in set increments. In alternative embodiments, the system 200 can be provided without the plunger actuator 216 and the plunger 210 can moved manually.

The system 200 can include a sensor 218 that can sense forces associated with, or indicative of, a region at which the tip 204 of the needle 202 is located within a patient. As described further later, the forces sensed by the sensor 218 can be used to infer whether the tip 204 is positioned within a target region of the patient, such as a suprachoroidal space between the sclera and the choroid. The sensor 218 can sense one or more aspects of the syringe 206 to sense the forces associated with, or indicative of, the region at which the tip 204 of the needle 202 is located within a patient. For example, in embodiments, the sensor 218 can be a pressure sensor that can sense pressures indirectly associated with the ejection of the medicament (e.g., backpressure exerted on the plunger 210 as the plunger 210 is moved distally towards the needle 202) or sense pressures directly associated with the medicament within the barrel 208. Additionally, or alternatively, the sensor 218 can directly or indirectly sense the force experienced by the tip 204 of the needle 202.

In embodiments, the system 200 can include a human machine interface (HMI) 220, which can facilitate human interaction with the system 200. The HMI 220 can include a button, a speaker, a light, a display, any of the other aspects described later in reference to the HMI 708, or combinations thereof, among other possibilities.

In embodiments, the system 200 can include a controller 222 that can control and/or communicate with various aspects of the system 200. For example, in embodiments, the controller 222 can communicate with and/or control aspects of the operation of the needle actuator 214, the plunger actuator 216, the sensor 218, the HMI 220, or combinations thereof, among other possibilities. The controller 222 can include any of the aspects described later in reference to the computing system 700.

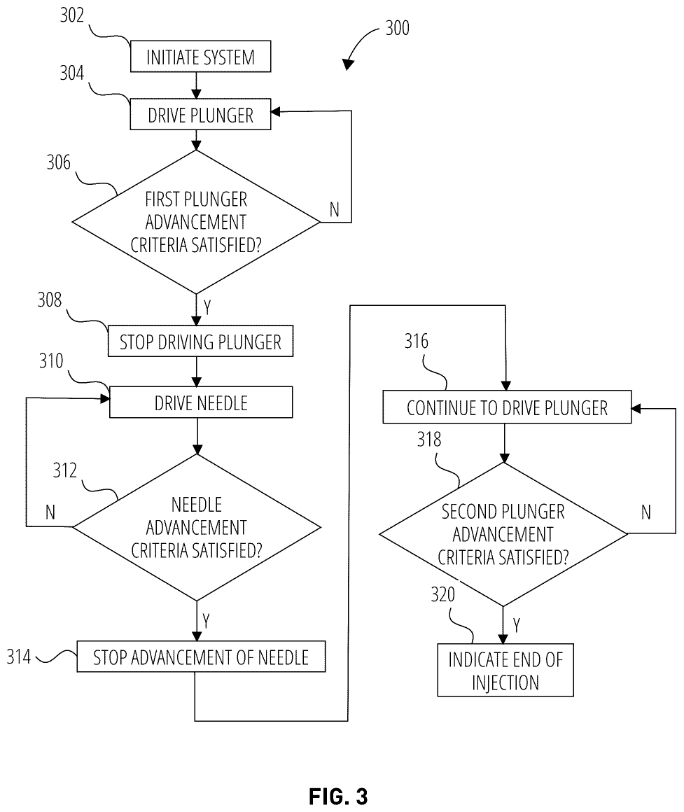

FIG. 3 shows a process 300 of injecting medicament into a target region. The process 300 can be implemented with embodiments of the system 200. For example, the process 300 can be implemented with embodiments of the system 200 that include both the needle actuator 214 and the plunger actuator 216.

The process 300 can begin at step 302, at which the system 200 can be initiated. Initiating the system 200 can include activating the controller 222 to automatically begin the process 300. Initiation of the system 200 at step 302 can occur in response to clinician input into the system 200, via the HMI 220, such as by pushing a button, flipping a switch, sending a signal from a remote device such as a smartphone, among other possibilities. The clinician can initiate the system 200 after engaging the tip 204 of the needle 202 with the target tissue. In embodiments, the target tissue can be scleral tissue of an eye and the process 300 can be implemented to inject medicament into a target region of the eye such as the suprachoroidal space between the sclera and the choroid. It is to be understood that the process 300 (as well as the process 400, the process 500, and the process 600 described later) and the system 200 are not limited to use with eye tissue and could alternatively be implemented to inject medicament within other target regions of a patient, such as for example an epidural space, among other possibilities. Accordingly, although the remainder of the process 300 will be described in light of medicament injection into an eye, and specifically into a target region of the eye corresponding to the suprachoroidal space, the process 300 is not limited to this example application.

The process 300 can include, at step 304, driving the plunger 210 distally towards the needle 202. Driving the plunger 210 distally towards the needle 202 can be performed automatically by the system 200 in response to the clinician initiating the system 200 via the HMI 220. For example, the plunger actuator 216 can automatically drive the plunger 210 distally towards the needle 202 in response to the clinician initiating the system 200 via the HMI 220. In embodiments, the power delivered to the plunger 210 via the plunger actuator 216 can be controlled (e.g., by the controller 222) to prevent or minimize dimpling of the sclera (and pain associated therewith) and to reduce or eliminate medicament injection into sclera tissue. It is to be understood that the power delivered to the plunger actuator 216 can depend on a number of factors including, for example, the diameter of the needle 202, feedback from the sensor 218 (e.g., backpressure exerted on the plunger 210), among other possibilities.

The process 300 can include, at step 306, determining, via the controller 222, whether a first plunger advancement criteria is satisfied. Determining satisfaction of the first plunger advancement criteria can involve comparing sensed backpressures exerted on the plunger 210, sensed by the sensor 218, with a threshold backpressure or range of threshold backpressures. The threshold backpressure or threshold range of backpressures can be a pressure sufficient to inject medicament within the target region (e.g., the suprachoroidal space), but insufficient to inject medicament within the tissue that the needle 202 extends through to reach the target region (e.g., the sclera). For example, the threshold back pressure can be between 5 kPa and 28 kPa, between 8 kPa and 20 kPa, among other possibilities. If the sensed backpressure exerted on the plunger 210 meets or exceeds the threshold backpressure or is within a range of threshold back pressures, the process 300 can determine that the first plunger advancement criteria is satisfied. If the sensed backpressure exerted on the plunger 210 fails to meet or exceed the threshold backpressure, fails to fall within the range of threshold backpressures, and/or is outside the range of threshold backpressures, the process 300 can determine that the first plunger advancement criteria is not satisfied.

If it is determined at step 306 that the first plunger advancement criteria is satisfied, the process 300 can advance to step 308, at which driving of the plunger 210 can be stopped. Stopping driving of the plunger 210 can prevent further increase of the pressure on the medicament, which can reduce or eliminate the risk of injecting the medicament into a region (e.g., within the sclera) other than the target region and can reduce or eliminate dimpling. Dimpling can be reduced or eliminated by removing the forces acting on the plunger 210 that would, via the syringe 206 and/or the needle 202, also act on the surface of the eye. Driving of the plunger 210 can be automatically stopped at step 308. For example, the controller 222 can deactivate the plunger actuator 216 to automatically stop the plunger 210 in response to determining that the needle advancement criteria is satisfied. In embodiments, automatically stopping the plunger 210 can include restricting at least proximal movement of the plunger 210, and in some embodiments, restricting both proximal and distal movement. Movement of the plunger 210 can be restricted with a lock or other mechanical aspect of the plunger actuator 216 and/or by powering the plunger actuator 216 to hold the backpressure at the threshold backpressure or range of threshold backpressures. The backpressure hold can be automatic in response to powering the plunger actuator 216. The “auto-hold” function can allow a user to release their finger from the plunger 210 once it is stopped. If it is determined at step 306 that the first plunger advancement criteria is not satisfied, the process 300 can repeat step 304 and step 306 and continue to drive the plunger 210 until the first plunger advancement criteria is satisfied. Step 304 and step 306 can be performed in parallel.

The process 300 can include, at step 310, driving the needle 202. When the clinician initiates the system 200 at step 302 after engaging the tip 204 of the needle 202 with the sclera, driving the needle 202 at step 310 can cause the tip 204 to penetrate into the sclera. This step can allow for the needle 202 to be positioned in the proper location adjacent to or within the sclera prior to delivering the medicament. Driving the needle 202 can be performed automatically by the system 200 after driving the plunger 210 is stopped at step 308. In embodiments, the power delivered to the needle 202, via the needle actuator 214, to move the needle 202 relative the barrel 208 can be controlled (e.g., by the controller 222) to prevent or minimize dimpling of the sclera (and pain associated therewith). In embodiments, controlling the power delivered to the needle actuator 214 can indirectly control a rate of advancement of the tip 204 into the sclera to limit or prevent over insertion of the tip 204 beyond the target region, e.g., the suprachoroidal space. It is to be understood that the power delivered to the needle actuator 214 can depend on a number of factors including, for example, feedback from the sensor 218 (e.g., sensed backpressure exerted on the plunger 210), the diameter of the needle 202, average or typical thicknesses of tissue (e.g., the sclera), response times of various aspects of the system 200 including controller 222 processing times and/or sensor 218 measurement intervals, among other possibilities. Driving the needle 202 at step 310 can occur after the plunger 210 is stopped at step 308. Performing step 310 after step 308 can be advantageous because, as described later, driving the needle 202 can be a function of the backpressure of the plunger 210, and achieving the threshold backpressure or threshold range of backpressures before driving the needle 102 at step 310 can improve the accuracy of this control.

The process 300 can include, at step 312, determining, via the controller 222, whether a needle advancement criteria is satisfied. Determining satisfaction of the needle advancement criteria can be a function of data collected from the sensor 218. For example, in embodiments the sensor 218 can collect data directly or indirectly associated with a backpressure of the plunger 210 that resists distal advancement of the plunger 210. When the target region is a cavity, such as the suprachoroidal space, the backpressure exerted on the plunger 210 can drop when the tip 204 of the needle 202 passes through the sclera and enters the suprachoroidal space because the medicament can more easily flow from the syringe 206 once the tip 204 enters the suprachoroidal space. In such embodiments, satisfaction of the needle advancement criteria can occur when the sensed backpressure exerted on the plunger 210 drops to a pressure indicative of or associated with the target region, e.g., the suprachoroidal space. For example, satisfaction of the needle advancement criteria can occur in response to one or more successive sensed backpressure measurements below the threshold backpressure or outside of (e.g., below) the range of threshold backpressures, as described previously.

If the needle advancement criteria is satisfied at step 312, the process 300 can proceed to step 314 at which advancement of the needle 202 can be stopped. Stopping advancement of the needle 202 at step 314 can be automatically controlled by the controller 222. For example, the controller 222 can stop directing power to the needle actuator 214 and can lock the needle 202 in place with a lock of the system 200. The lock can be an aspect of the needle actuator 214 or a distinct component of the system 200. If the needle advancement criteria is not satisfied at step 312, the process 300 can repeat step 310 and step 312 and can continue to drive the needle 202 until the needle advancement criteria is satisfied. Step 310 and step 312 can be performed in parallel.

After step 314, the process 300 can proceed to step 316, at which the plunger 210 can continue to be driven distally towards the needle 202. Continuing to drive the plunger 210 at step 316 can cause the medicament to be injected into the target region, e.g., the suprachoroidal space. This is because advancement of the needle 202 can be stopped at step 314 in response to the determination at step 312 that the needle advancement criteria was satisfied, which is indicative of the tip 204 having reached the target region, e.g., the suprachoroidal space. Continuing to drive the plunger 210 distally towards the needle 202 can be performed automatically by the system 200 in response to reaching or completing step 314. For example, the plunger actuator 216 can automatically drive the plunger 210 distally towards the needle 202 in response to reaching or completing step 314, as described previously in step 304. The power delivered to the plunger actuator 216 at step 316 can be the greater than, less than, or the same as the power delivered to the plunger actuator 216 at step 304.

The process 300 can include, at step 318, determining, via the controller 222, whether a second plunger advancement criteria is satisfied. Determining satisfaction of the second plunger advancement criteria can be a function of forces sensed from the sensor 218. For example, in embodiments, the sensor 218 can sense backpressures exerted on the plunger 210 that resists distal advancement of the plunger 210. When the plunger 210 is advanced with the tip 204 of the needle 202 in the target region, e.g., the suprachoroidal space, medicament can be injected within the target region and the backpressure on the plunger 210 can decrease until the plunger 210 reaches the distal end 226 of the barrel 208. Once the plunger 210 reaches the distal end 226 of the barrel 208, the backpressure can rapidly increase. The controller 222 can determine that the second plunger advancement criteria is satisfied when the sensed backpressure exerted on the plunger 210 rapidly increases, since this can be indicative of the plunger 210 contacting the distal end 226 of the barrel 208. If the second plunger advancement criteria is not satisfied at step 318, the process 300 can repeat step 316 and step 318 until the second plunger advancement criteria is satisfied. Step 316 and step 318 can be performed in parallel. The procedures above allow injection of a full dose of medicament with minimum dead volume of medicament left in the syringe 206.

If the second plunger advancement criteria is satisfied at step 318, the process 300 can proceed to step 320, at which system 200 can indicate that the injection has ended. Indicating that the injection has ended at step 320 can include, for example, providing audio, visual, or tactile feedback via the HMI 220. Step 320 can further include automatically deactivating powered components of the system 200, such as the plunger actuator 216.

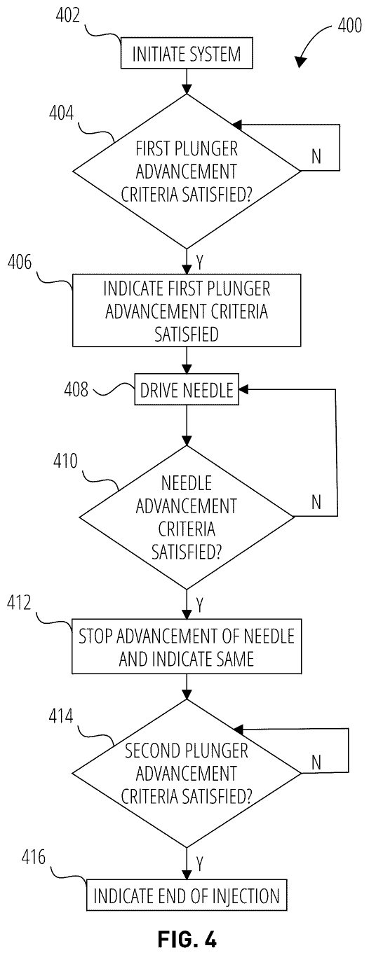

FIG. 4 shows a process 400 of injecting medicament into a target region of a patient. The process 400 can be implemented with embodiments of the system 200. For example, the process 400 can be implemented with embodiments of the system 200 that include a needle actuator 214 that can be automatically controlled, but without a plunger actuator 216 subject to automatic control from the controller 222 or with a plunger actuator 216 that can be both manually and automatically controlled. In such embodiments, the plunger 210 can be manually controlled by a clinician, while the needle actuator 214 can be automatically operated by the system 200 in accordance with, for example, the process 400 described as follows.

The process 400 can begin at step 402, at which the system 200 can be initiated. Initiating the system 200 can include activating the controller 222 to automatically begin the process 400. Initiation of the system 200 at step 402 can occur in response to clinician input into the system 200, via the HMI 220, such as by pushing a button, flipping a switch, sending a signal from a remote device such as a smartphone, among other possibilities. The clinician can initiate the system 200 after engaging the tip 204 of the needle 202 with target tissue. In embodiments, the target tissue can be scleral tissue and the process 400 can be implemented to inject medicament into a target region of the eye such as the suprachoroidal space between the sclera and the choroid.

After the system 200 is initiated at step 402, a clinician can manually drive the plunger 210 distally towards the needle 202.

The process 400 can include, at step 404, determining, via the controller 222, whether a first plunger advancement criteria is satisfied. Determining satisfaction of the first plunger advancement criteria can include any of the aspects described previously in reference to analogous step 306 of the process 300. If it is determined at step 404 that the first plunger advancement criteria is satisfied, the process 400 can advance to step 406, at which the system 200 can indicate, via the HMI 220, that the first plunger advancement criteria is satisfied. Indicating that the first plunger advancement criteria is satisfied can include, for example, providing audio, visual, or tactile feedback via the HMI 220. Indicating that the first plunger advancement criteria can encourage a clinician to stop driving the plunger 210 and hold the position of the plunger 210. If it is determined at step 404 that the first plunger advancement criteria is not satisfied, the process 400 can repeat step 404 until the first plunger advancement criteria is satisfied. Step 404 and step 406 can be optional. When the process 400 is performed without the step 404 and step 406, a clinician can attempt to achieve the threshold backpressure or range of threshold backpressures by feel and without feedback from the system 200.

The process 400 can include, at step 408, driving the needle 202. Driving the needle 202 at step 408 can include any of the aspects described previously in reference to analogous step 310 of the process 300. When the clinician initiates the system 200 at step 402 after engaging the tip 204 of the needle 202 with the sclera, driving the needle 202 at step 408 can cause the tip 204 to penetrate into the sclera. Driving the needle 202 can be performed automatically by the system 200 in response to clinician input. For example, a clinician can trigger step 408 via the HMI 220 by, for example, pushing a button, flipping a switch, sending a signal from a remote device such as a smartphone, among other possibilities. The clinician can be trained or encouraged to trigger step 408 after the system 200 determines the first plunger advancement criteria is satisfied at step 404 or after the clinician estimates that the threshold backpressure or range of threshold backpressures has been achieved.

The process 400 can include, at step 410, determining, via the controller 222, whether a needle advancement criteria is satisfied. Determining satisfaction of the needle advancement criteria can include any of the aspects described previously in reference to analogous step 312 of the process 300. If the needle advancement criteria is satisfied at step 410, the process 400 can proceed to step 412 at which advancement of the needle 202 can be stopped. Stopping advancement of the needle 202 at step 412 can include any of the aspects described previously in reference to analogous step 314 of the process 300. Additionally, step 412 can include indicating that the needle advancement criteria is satisfied by, for example, providing audio, visual, or tactile feedback via the HMI 220. Indicating that the needle advancement criteria is satisfied can encourage a clinician to manually drive the plunger 210 and inject the medicament into the target region, e.g., the suprachoroidal space. If the needle advancement criteria is not satisfied at step 410, the process 400 can repeat step 408 and step 410 and can continue to drive the needle 202 until the needle advancement criteria is satisfied. Step 408 and step 410 can be performed in parallel.

The process 400 can include, at step 414, determining, via the controller 222, whether a second plunger advancement criteria is satisfied. Determining satisfaction of the second plunger advancement criteria can include any of the aspects described previously in reference to analogous step 318 of the process 300. If the second plunger advancement criteria is not satisfied at step 414, the process 400 can repeat step 414 until the second plunger advancement criteria is satisfied.

If the second plunger advancement criteria is satisfied at step 414, the process 400 can proceed to step 416, at which system 200 can indicate that the injection has ended. Indicating that the injection has ended at step 416 can include, for example, providing audio, visual, or tactile feedback via the HMI 220. It is to be appreciated that the indication that the injection has ended can be distinguishable from the indication that the first plunger advancement criteria is satisfied, which can help the clinician distinguish between indications. Step 416 can further include automatically deactivating powered components of the system 200.

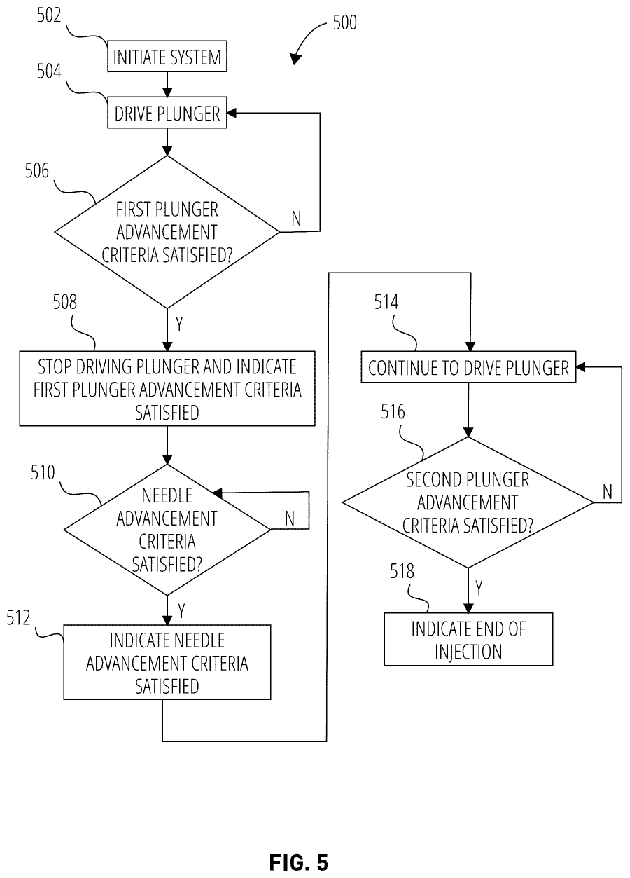

FIG. 5 shows a process 500 of injecting medicament into a target region of a patient. The process 500 can be implemented with embodiments of the system 200. For example, the process 500 can be implemented with embodiments of the system 200 that include a plunger actuator 216 that can be automatically controlled, but without a needle actuator 214 (e.g., with a needle 202 that is fixed relative to the barrel 208) or with a needle actuator 214 that can be both manually and automatically controlled. In such embodiments, a depth at which the needle 202 is inserted into the patient can be manually controlled (with or without the assistance of a manual needle actuator 214) by a clinician, while the plunger actuator 216 can be automatically operated by the system 200 in accordance with, for example, the process 500 described as follows.

The process 500 can begin at step 502, at which the system 200 can be initiated. Initiating the system 200 can include activating the controller 222 to automatically begin the process 500. Initiation of the system 200 at step 502 can occur in response to clinician input into the system 200, via the HMI 220, such as by pushing a button, flipping a switch, sending a signal from a remote device such as a smartphone, among other possibilities. The clinician can initiate the system 200 after engaging the tip 204 of the needle 202 with target tissue. In embodiments, the target tissue can be sclera tissue and the process 500 can be implemented to inject medicament into a target region of the eye such as the suprachoroidal space between the sclera and the choroid.

The process 500 can include, at step 504, driving the plunger 210 distally towards the needle 202. Driving the plunger 210 at step 504 can include any of the aspects previously described in reference to analogous step 304 of the process 300.

The process 500 can include, at step 506, determining, via the controller 222, whether a first plunger advancement criteria is satisfied. Determining satisfaction of the first plunger advancement criteria can include any of the aspects described previously in reference to analogous step 306 of the process 300. If it is determined at step 506 that the first plunger advancement criteria is satisfied, the process 500 can advance to step 508, at which driving of the plunger 210 can be stopped. If it is determined at step 506 that the first plunger advancement criteria is not satisfied, the process 500 can repeat step 504 and step 506 and continue to drive the plunger 210 until the first plunger advancement criteria is satisfied. Step 504 and step 506 can be performed in parallel.

Stopping driving the plunger 210 at step 508 can include any of the aspects described previously in reference to analogous step 308 of the process 300. Step 508 can also include indicating that the first plunger advancement criteria is satisfied. For example, the system 200 can indicate, via the HMI 220, that the first plunger advancement criteria is satisfied by, for example, providing audio, visual, or tactile feedback via the HMI 220. Indicating that the first plunger advancement criteria is satisfied can encourage a clinician to manually drive the needle 202 through the target tissue (e.g., the sclera) and towards the target region (e.g., the suprachoroidal space). In embodiments, the needle 202 can be fixed relative to the barrel 208 and the clinician can manually drive the entire syringe 206 to extend the needle 202 through the target tissue to the target region. Alternatively, the needle 202 can be movable relative to the barrel 208 via needle actuator 214 (e.g., a ratchet, among other possibilities), which can be manually actuated/manually powered by the clinician to manually drive the needle 202 relative to the barrel 208 through the target tissue to the target region.

The process 500 can include, at step 510, determining, via the controller 222, whether a needle advancement criteria is satisfied. Determining satisfaction of the needle advancement criteria can include any of the aspects previously described in reference to analogous step 312 of the process 300. If it is determined at step 510 that the needle advancement criteria is satisfied, the process 500 can advance to step 512, at which the system 200 can indicate, via the HMI 220, that the needle advancement criteria is satisfied. Indicating that the needle advancement criteria is satisfied can include, for example, providing audio, visual, or tactile feedback via the HMI 220. Indicating that the needle advancement criteria is satisfied can encourage a clinician to stop manually advancing the needle 202 and hold the needle 202 in position, which can be associated with the target region since the needle advancement criteria is satisfied. It is to be appreciated that the indication that the needle advancement criteria is satisfied can be distinguishable from the indication that the indication that the first plunger advancement criteria is satisfied, which can help the clinician distinguish between indications. If it is determined at step 510 that the needle advancement criteria is not satisfied, the process 500 can repeat step 510 until the needle advancement criteria is satisfied.

The process 500 can include, at step 514, continuing to drive the plunger 210 distally towards the needle 202 to inject the medicament. Continuing to drive the plunger 210 at step 514 can include any of the aspects described previously in reference to analogous step 316 of the process 300. Step 514 can occur automatically in response to step 512. Alternatively, step 514 can occur in response to input from the clinician via the HMI 220 (e.g., pushing a button, flipping a switch, sending a signal from a remote device such as a smartphone, among other possibilities), which can be advantageous because it can provide a mechanism for the clinician to affirm that they are holding the needle in place, e.g., within the target region, before the injection commences to increase the amount of medicament injected within the target region.

The process 500 can include, at step 516, determining, via the controller 222, whether a second plunger advancement criteria is satisfied. Determining satisfaction of the second plunger advancement criteria can include any of the aspects described previously with respect to analogous step 318 of the process 300. If the second plunger advancement criteria is not satisfied at step 516, the process 500 can repeat step 514 and step 516 until the second plunger advancement criteria is satisfied. Step 514 and step 516 can be performed in parallel.

If the second plunger advancement criteria is satisfied at step 516, the process 500 can proceed to step 518, at which the system 200 can indicate that the injection has ended. Indicating that the injection has ended at step 518 can include, for example, providing audio, visual, or tactile feedback via the HMI 220. It is to be appreciated that the indication that the injection has ended can be distinguishable from the indication that the first plunger advancement criteria is satisfied and distinguishable from the indication that the needle advancement criteria is satisfied, which can help the clinician distinguish between the indications. Step 518 can further include automatically deactivating powered components of the system 200, such as the plunger actuator 216.

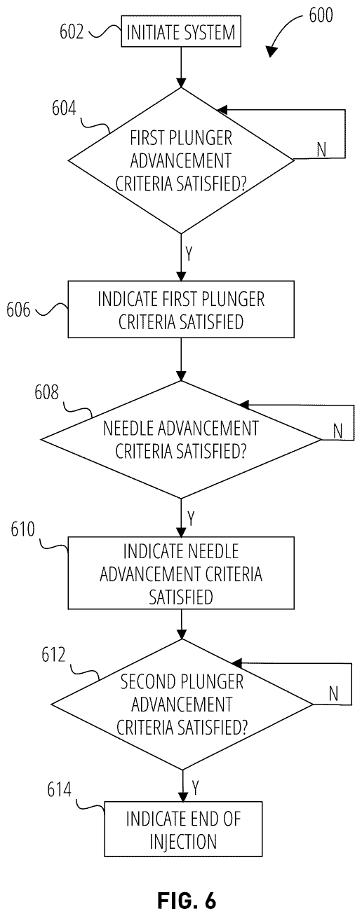

FIG. 6 shows a process 600 of injecting medicament into a target region of a patient. The process 600 can be implemented with embodiments of the system 200. For example, the process 600 can be implemented with embodiments of the system 200 with a plunger actuator 216 that can be manually controlled or without a plunger actuator 216, and with a needle actuator 214 that can be manually controlled or without a needle actuator 214 (e.g., with a needle 202 fixed relative to the barrel 208). In such embodiments, both a depth at which the needle 202 is inserted into the patient can be manually controlled (with or without the assistance of a manual needle actuator 214) by a clinician and the plunger 210 can be manually driven by the clinician. The process 600 can provide guidance to the clinician, via the system 200, during this manual control.

The process 600 can begin at step 602, at which the system 200 can be initiated. Initiating the system 200 can include activating the controller 222 to automatically begin the process 600. Initiation of the system 200 at step 602 can occur in response to clinician input into the system 200, via the HMI 220, such as by pushing a button, flipping a switch, sending a signal from a remote device such as a smartphone, among other possibilities. The clinician can initiate the system 200 after engaging the tip 204 of the needle 202 with target tissue. In embodiments, the target tissue can be sclera tissue and the process 600 can be implemented to inject medicament into a target region of the eye, such as the suprachoroidal space between the sclera and the choroid.

After the system 200 is initiated at step 602, a clinician can manually drive the plunger 210 distally towards the needle 202.

The process 600 can include, at step 604, determining, via the controller 222, whether a first plunger advancement criteria is satisfied. Determining satisfaction of the first plunger advancement criteria can include any of the aspects described previously in reference to analogous step 306 of the process 300. If it is determined at step 604 that the first plunger advancement criteria is not satisfied, the process 600 can repeat step 604 until the first plunger advancement criteria is satisfied.

If it is determined at step 604 that the first plunger advancement criteria is satisfied, the process 600 can advance to step 606, at which the system 200 can indicate that the first plunger advancement criteria is satisfied. For example, the system 200 can indicate, via the HMI 220, that the first plunger advancement criteria is satisfied by, for example, providing audio, visual, or tactile feedback via the HMI 220. Indicating that the first plunger advancement criteria is satisfied can encourage a clinician to manually drive the needle 202 through the target tissue (e.g., the sclera) and towards the target region (e.g., the suprachoroidal space). In embodiments, the needle 202 can be fixed relative to the barrel 208 and the clinician can manually drive the entire syringe 206 to extend the needle 202 through the target tissue to the target region. Alternatively, the needle 202 can be movable relative to the barrel 208 via needle actuator 214 (e.g., a ratchet, among other possibilities), which can be manually actuated/manually powered by the clinician to manually drive the needle 202 relative to the barrel 208 through the target tissue to the target region.

The process 600 can include, at step 608, determining, via the controller 222, whether a needle advancement criteria is satisfied. Determining satisfaction of the needle advancement criteria can include any of the aspects previously described in reference to analogous step 312 of the process 300. If it is determined at step 608 that the needle advancement criteria is satisfied, the process 600 can advance to step 610, at which the system 200 can indicate, via the HMI 220, that the needle advancement criteria is satisfied. Indicating that the needle advancement criteria is satisfied can include, for example, providing audio, visual, or tactile feedback via the HMI 220. Indicating that the needle advancement criteria is satisfied can encourage a clinician to stop manually advancing the needle 202 and hold the needle 202 in position, which can be associated with the target region since the needle advancement criteria is satisfied. Moreover, indicating that the needle advancement criteria is satisfied can encourage a clinician to manually drive the plunger 210 and inject the medicament into the target region, e.g., the suprachoroidal space. It is to be appreciated that the indication that the needle advancement criteria is satisfied can be distinguishable from the indication that the first plunger advancement criteria is satisfied, which can help the clinician distinguish between the indications. If it is determined at step 608 that the needle advancement criteria is not satisfied, the process 600 can repeat step 608 until the needle advancement criteria is satisfied.

The process 600 can include, at step 612, determining, via the controller 222, whether a second plunger advancement criteria is satisfied. Determining satisfaction of the second plunger advancement criteria can include any of the aspects described previously with respect to analogous step 318 of the process 300. If the second plunger advancement criteria is not satisfied at step 612, the process 600 can repeat step 612 until the second plunger advancement criteria is satisfied.

If the second plunger advancement criteria is satisfied at step 612, the process 600 can proceed to step 614, at which the system 200 can indicate that the injection has ended. Indicating that the injection has ended at step 614 can include, for example, providing audio, visual, or tactile feedback via the HMI 220. It is to be appreciated that the indication that the injection has ended can be distinguishable from the indication that the first plunger advancement criteria is satisfied and from the indication that the needle advancement criteria is satisfied, which can help the clinician distinguish between the indications. Step 614 can further include automatically deactivating powered components of the system 200.

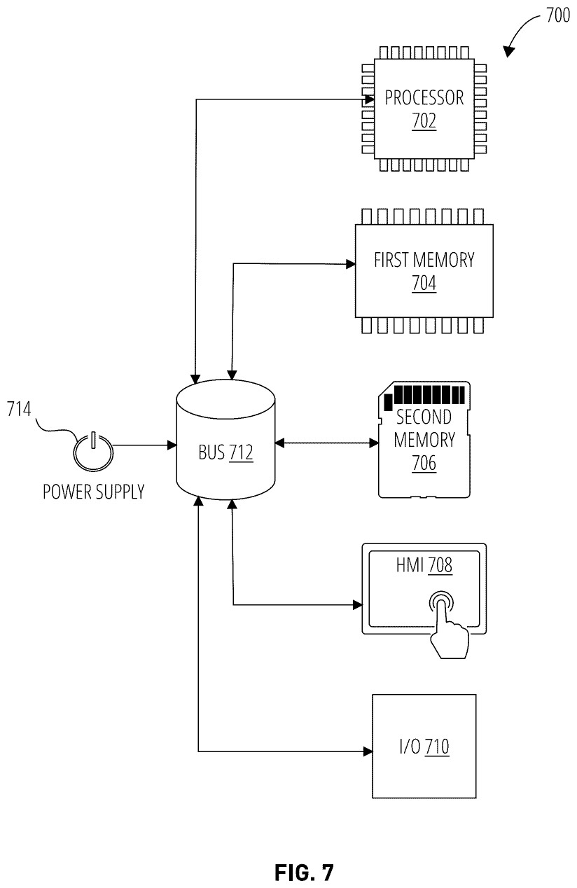

FIG. 7 shows an example computing system 700 in accordance with aspects of the disclosure. Various embodiments of this disclosure can include or be implemented together with various aspects of the computing system 700. For example, aspects of the controller 222, the needle actuator 214, the plunger actuator 216, the sensor 218, and the HMI 220 can be implemented together with or can include some or each of the features, structures, relationships described as follows in reference to the computing system 700.

The computing system 700 can include a processor 702, or CPU, which can execute instructions from programs and can perform basic arithmetic, logic, control, and input/output operations. The processor 702 can be a general purpose processor, a special processor, among other possibilities. The computing system 700 can include a first memory 704 (e.g., a random access memory or other computer readable storage medium) that can temporarily store data and instructions that the processor 702 can quickly utilize to improve multitasking and the speed that the processor 702 can execute tasks. The computing system 700 can include a second memory 706 that can store data permanently (e.g., until instructed to delete the data) and/or for longer than the first memory 704 to allow the data to be accessible after the computing system 700 has been turned off. The second memory 706 can be any non-transitory computer readable medium capable of long term data storage including, for example, a hard disk drive, a removable storage drive (e.g., a flash memory, a universal serial bus drive, etc.), combinations thereof, among other possibilities. In embodiments, the second memory 706 can include other structures or features for allowing computer programs or other instructions to be loaded into computing system 700 such as, for example, a removable storage unit, an interface, a program cartridge and cartridge interface, a removable memory chip and associated socket, combinations thereof, among other possibilities.

The computing system 700 can include an HMI 708, or human machine interface, which can include any combination of features to allow a person to interact with or control the computing system 700. The HMI 220 can be an embodiment of the HMI 708. The HMI 708 can include, for example, a display, a touchscreen, a keyboard, a mouse, a track pad, a button, a switch, a dial, a speaker, a microphone, a light, combinations thereof, among other possibilities. The computing system 700 can include an I/O 710, or input/output, which can facilitate communication between the computing system 700 and the outside world by transferring data to and from external devices, systems, or users. The I/O 710 can include, for example, a network interface (such as an Ethernet card), a modem, a communication port, combinations thereof, among other possibilities. Software and data transferred via the I/O 710 can be in the form of signals, which can be electronic, electromagnetic, optical, or other signals capable of being received by I/O 710. Such signals can be provided to the I/O 710 via a communication path that can carry signals. Such a communication path can be implemented using wire, cable (e.g., fiber optics), wirelessly (e.g., via a cellular link, an RF link, etc.), combinations thereof, among other possibilities. In embodiments, such a communication path can be an aspect of the computing system 700.

The computing system 700 can include a bus 712, or other functionally equivalent structure, capable of transferring data between the various components of the computing system 700 including, e.g., the processor 702, the first memory 704, the second memory 706, the HMI 220/HMI 708, the I/O 710, the needle actuator 214, the plunger actuator 216, the sensor 218, combinations thereof, among other possibilities.

The computing system 700 can include or can be implemented with a power supply 714 that can power some or all of the powered components of the computing system 700 or any of the powered components described herein. The power supply 714 can be local, such as a battery, a capacitor, a photovoltaic cell, a fuel cell, combinations thereof, among other possibilities. Additionally, or alternatively, the power supply 714 can be distributed over a grid, or the like, from a remote source.

Various aspects of this disclosure (e.g., the process 300, the process 400, the process 500, the process 600 and capabilities described previously) can be embodied as computer programs, computer control logic, databases, combinations thereof, among other possibilities. Such aspects can be stored on various non-transitory computer readable media (e.g., the first memory 704, the second memory 706, etc.) and can be executed by the processor 702. Such aspects, when executed, can enable the processor 702 (alone or together with other aspects of the computing system 700) to implement the various processes and capabilities, described previously.

It will be appreciated that the foregoing description provides examples of the invention. However, it is contemplated that other implementations of the invention may differ in detail from the foregoing examples. All references to the invention or examples thereof are intended to reference the particular example being discussed at that point and are not intended to imply any limitation as to the scope of the invention more generally. All language of distinction and disparagement with respect to certain features is intended to indicate a lack of preference for those features, but not to exclude such from the scope of the invention entirely unless otherwise indicated.

What is claimed is:

1. A system for injecting medicament into a target region, the system comprising:
a syringe comprising:
a barrel configured to contain the medicament;
a plunger movable within the barrel; and
a needle in fluid communication with the barrel, the needle comprising a tip;
a sensor configured to sense a backpressure exerted on the plunger; and
a controller configured, when the tip of the needle extends through a target tissue and into the target region, to determine satisfaction of a needle advancement criteria based on the sensed backpressure.

2. The system of claim 1, wherein the controller is configured to determine satisfaction of the needle advancement criteria based on the sensed backpressure changing from within a range of threshold backpressures to outside the range of threshold backpressures.

3. The system of claim 2, wherein the controller is configured to determine satisfaction of the needle advancement criteria based on the sensed backpressure falling from within the range of the range of threshold backpressures to below the range of threshold backpressures.

4. The system of claim 2, wherein:
the system further comprises a needle actuator configured to drive the needle distally relative to the barrel, and
the controller is configured to control the needle actuator based on satisfaction of the needle advancement criteria.

5. The system of claim 4, wherein:
the controller is configured, when the tip of the needle is in contact with the target tissue and the sensed backpressure is within the range of threshold backpressures, to determine that the needle advancement criteria is not satisfied, and
the controller is configured, in response to the determination that the needle advancement criteria is not satisfied, to control the needle actuator to drive the needle distally relative to the barrel until the needle advancement criteria is satisfied.

6. The system of claim 4, wherein:
the system further comprises a human machine interface, and
the controller is configured, in response to a determination of satisfaction of the needle advancement criteria, to control the human machine interface to indicate that the needle advancement criteria is satisfied.

7. The system of claim 1, wherein the controller is configured, when the tip of the needle is in contact with the target tissue and the sensed backpressure is within a range of threshold backpressures, to determine satisfaction of a plunger advancement criteria.

8. The system of claim 7, wherein:
the system further comprises a plunger actuator configured to drive the plunger to cause the plunger to move distally within the barrel, and
the controller is configured to control the plunger actuator based on satisfaction of the plunger advancement criteria.

9. The system of claim 8, wherein:
the controller is configured, when the tip of the needle is in contact with the target tissue and the sensed backpressure is outside the range of threshold backpressures, to determine that the plunger advancement criteria is not satisfied, and
the controller is configured, in response to the determination that the plunger advancement criteria is not satisfied, to control the plunger actuator to drive the plunger distally until the plunger advancement criteria is satisfied.

10. The system of claim 9, wherein:
the system further comprises a needle actuator configured to drive the needle distally relative to the barrel, and
the controller is configured to control the needle actuator based on satisfaction of the needle advancement criteria.

11. The system of claim 10, wherein:
the controller is configured to determine satisfaction of the needle advancement criteria based the sensed backpressure changing from being within the range of threshold backpressures to outside the range of threshold backpressures,
the controller is configured, when the tip of the needle is in contact with the target tissue and the sensed backpressure is within the range of threshold backpressures, to determine that the needle advancement criteria is not satisfied, and
the controller is configured, in response to the determination that the needle advancement criteria is not satisfied, to control the needle actuator to drive the needle distally relative to the barrel until the needle advancement criteria is satisfied.

12. The system of claim 11, wherein:
the plunger advancement criteria is a first plunger advancement criteria, and
the controller is configured to determine, when the tip of the needle is within the target region and the plunger contacts a distal end of the barrel, satisfaction of a second plunger advancement criteria in response to a rise in the sensed backpressure.

13. The system of claim 12, wherein:
the system further comprises a human machine interface, and
the controller is configured, in response to a determination of satisfaction of the at least one of the needle advancement criteria, the first plunger advancement criteria, or the second plunger advancement criteria, to control the human machine interface to indicate that the at least one of the needle advancement criteria, the first plunger advancement criteria, or the second plunger advancement criteria is satisfied.

14. The system of claim 2, wherein the range of threshold backpressures is between 5 kPa and 28 kPa.

15. The system of claim 1, wherein the target tissue is sclera tissue and the target region is a suprachoroidal space.

16. A method of injecting medicament into a target region, the method comprising:
determining, based on sensed backpressure associated with a back pressure exerted on a plunger of a syringe, that a needle advancement criteria is not satisfied when a tip of a needle of the syringe extends into a target tissue;
driving, in response to the determination that the needle advancement criteria is not satisfied, the needle into the target tissue until the tip of the needle reaches a target region,
determining, based on the sensed backpressure exerted on the plunger of the syringe, that the needle advancement criteria is satisfied after the tip of the needle is in the target region;
stopping, in response to the determination that the needle advancement criteria is satisfied, the driving of the needle; and
injecting the medicament within the target region.

17. The method of claim 16, wherein:
determining that the needle advancement criteria is not satisfied is based on the sensed backpressure being within a range of threshold backpressures, and
determining that the needle advancement criteria is satisfied is based on the sensed backpressure changing from within the range of threshold backpressures to outside of the range of threshold backpressures.

18. The method of claim 16, further comprising indicating that the needle advancement criteria is satisfied in response to determining that the needle advancement criteria is satisfied.

19. The method of claim 16, further comprising:
determining, based on the sensed backpressure exerted on the plunger of the syringe, before determining that the needle advancement criteria is not satisfied, that a plunger advancement criteria is not satisfied;
driving, in response to determining that the plunger advancement criteria is not satisfied, the plunger distally with the tip of the needle in contact with the target tissue; and
determining, based on the sensed backpressure exerted on the plunger of the syringe, after driving the plunger distally, that the plunger advancement criteria is satisfied; and
stopping, in response to determining that the plunger advancement criteria is satisfied, the driving of the plunger.

20. The method of claim 19, wherein:
determining that the plunger advancement criteria is not satisfied is based on the sensed backpressure exerted on the plunger of the syringe being outside a range of threshold backpressures, and
determining that the plunger advancement criteria is satisfied is based on the sensed backpressure exerted on the plunger of the syringe being within the range of threshold backpressures.

21. The method of claim 19, wherein:
driving of the plunger comprises controlling a plunger actuator to drive the plunger distally, and
stopping the driving of the plunger comprises controlling the plunger actuator to stop the drive of the plunger.

22. The method of claim 19, further comprising indicating that the plunger advancement criteria is satisfied in response to determining that the plunger advancement criteria is satisfied.
